(19) United States
(12) Patent Application Publication
(10) Pub. No.: US 2012/0020899 A1
(43) Pub. Date: Jan. 26, 2012

(54) ORAL COMPOSITIONS FOR TREATING TOOTH SENSITIVITY AND METHODS OF USE AND MANUFACTURE THEREOF

(75) Inventors: Lynette Zaidel, Cranford, NJ (US); Michael Precipe, Princeton Junction, NJ (US); Suman K. Chopra, Monroe, NJ (US)

(73) Assignee: Colgate-Palmolive Company, New York, NY (US)

(21) Appl. No.: 13/262,015

(22) PCT Filed: Apr. 1, 2010
(86) PCT No.: PCT/US10/29686

§ 371 Date: Sep. 29, 2011

Related U.S. Application Data
(60) Provisional application No. 61/165,802, filed on Apr. 1, 2009.

(51) Int. Cl. A61K 8/25; A61K 8/81; A61K 8/89; A61Q 11/00; A61K 8/64; A61K 8/98; A61K 8/44; A61K 8/18; A61K 8/60

(52) U.S. Cl. 424/52; 424/49; 424/54

(57) ABSTRACT

The invention encompasses oral care compositions comprising one or more active component and one or more adhesive polymers, which cause the active component to adhere to the tooth surface. In certain embodiments the active agent is an occlusion agent. The invention also encompasses methods of treating the teeth or a teeth surface with an active agent. In certain embodiments, the invention encompasses treating the teeth with an occlusion agent to prevent or alleviate tooth sensitivity.

### FIELD OF THE INVENTION

The invention encompasses oral care compositions comprising one or more active components and one or more bioadhesive polymers, which cause the active component to adhere to a tooth surface. In certain embodiments the active agent is an occlusion agent. The invention also encompasses methods of treating the teeth or a teeth surface with an active agent. In certain embodiments, the invention encompasses treating the teeth with an occlusion agent to prevent or alleviate tooth sensitivity.

### BACKGROUND OF THE INVENTION

There are certain situations where it is desirable to have prolonged contact of an oral care composition with teeth. For example, it may be desirable to treat or prevent, for example, xerostomia (dry mouth), tooth hypersensitivity, dental caries with levels of active agent for prolonged periods of time. This may be accomplished by the use of a dental tray, wherein a composition is applied to the dental tray, and then the composition and tray are applied to the teeth to be treated; however, this method is inconvenient, as the user is forced to retain the tray in their mouth during use, and thus the treatment time is limited by how long the user may retain the tray in their mouth.

This can also be achieved by using a tooth varnish; however, presently used tooth varnishes have the disadvantage of being multiphase, for example, as the active component is insoluble in the adhesive film forming phase, and the varnish may separate out into distinct phases. Additionally, components of the adhesive film forming phase may also separate into distinct phases over time. Users typically need to stir the varnish in order to mix the phases, which is time consuming and wasteful, as the varnish adheres to the mixing apparatus and is then discarded.

The inventors have developed a oral care product with improved efficacy, which incorporates orally adhesive polymers that increase product retention on the tooth surface.

### SUMMARY OF THE INVENTION

The compositions of the invention generally include one or more active components and one or more bioadhesive polymer components to allow the active material to adhere to one or more tooth surfaces.

In one embodiment, the invention encompasses oral care compositions including (i) one or more active components, for example, occlusion agents, an anti-caries agent, a fluoride source, an agent treat xerostomia, a desensitizing agent, and/or whitener or teeth bleach, bioactive glass (e.g., Novamin), arginine/calcium carbonate, arginine bicarbonate/calcium carbonate (e.g., Cavistat/PCC), and silice, for example, small particle silica (e.g., Sorbosil AC43 from Ineos) or combinations thereof and (ii) one or more bioadhesive or retentive polymers, for example, PEG/PPG copolymers (e.g., BASF Pluracare L1220), polyvinylmethylether/maleic acid copyolmer (e.g., Gantrez, ISP), cross-linked PVP (e.g. Polyplasdone, ISP), shellac (e.g., R49 Shellac, Mantrose-Hauser), and ester gum (e.g. Eastman Chemicals).

In another embodiment, the invention encompasses oral care compositions including (i) one or more occlusion agents and (ii) one or more bioadhesive or retentive polymers, for example, PEG/PPG copolymers (e.g., BASF Pluracare L1220), polyvinylmethylether/maleic acid copyolmer (e.g., Gantrez, ISP), cross-linked PVP (e.g., Polyplasdone, ISP), shellac (e.g., R49 Shellac, Mantrose-Hauser), and ester gum (e.g., Eastman Chemicals). In certain embodiments, the occlusion agent is bioactive glass, arginine/calcium carbonate, arginine bicarbonate/calcium carbonate (e.g., Cavistat/PCC), and small particle silica or combinations thereof.

The invention also encompasses methods of treating or preventing disorders of the oral cavity in a subject in need thereof.

Generally, the invention encompasses methods of treating or preventing disorders of the oral cavity in a subject in need thereof including administering to the oral cavity, specifically the teeth or a tooth surface an oral care composition of the invention. In various embodiments, the compositions for use in the methods of the invention include (i) one or more active components, for example, occlusion agents, an anti-caries agent, a fluoride source, an agent treat xerostomia, a desensitizing agent, and/or whitener or teeth bleach, bioactive glass (e.g., Novamin), arginine/calcium carbonate, arginine bicarbonate/calcium carbonate (e.g., Cavistat/PCC), and silica, for example, small particle silica (e.g., Sorbosil AC43 from Ineos) or combinations thereof and (ii) one or more bioadhesive or retentive polymers, for example, PEG/PPG copolymers (e.g., BASF Pluracare L1220), polyvinylmethylether/maleic acid copyolmer (e.g., Gantrez, ISP), cross-linked PVP (e.g., Polyplasdone, ISP), shellac (e.g., R49 Shellac, Mantrose-Hauser), and ester gum (e.g., Eastman Chemicals).

In one embodiment, the invention encompasses methods for treating dental hypersensitivity in a subject in need thereof comprising contacting one or more hypersensitive teeth with an effective amount of one or more occlusion agents and one or more bioadhesive polymers.

In another embodiment, the invention encompasses methods for at least partially occluding dentin tubules in a subject in need thereof comprising contacting said tubules with an effective amount of one or more occlusion agents and one or more bioadhesive polymers.

In another embodiment, the invention encompasses methods for preventing tooth decay in a subject in need thereof comprising contacting a tooth structure with an effective amount of one or more occlusion agents and one or more bioadhesive polymers.

In another embodiment, the invention encompasses methods for preventing incipient carries in a subject in need thereof comprising contacting a tooth structure with an effective amount of one or more occlusion agents and one or more bioadhesive polymers.

In another embodiment, the invention encompasses methods for remineralizing enamel in a subject in need thereof comprising contacting a tooth structure with an effective amount of one or more occlusion agents and one or more bioadhesive polymers.

In another embodiment, the invention encompasses methods for sealing fissures in tooth structure in a subject in need thereof comprising contacting a tooth structure with an effective amount of one or more occlusion agents and one or more bioadhesive polymers.

In another embodiment, the invention encompasses methods for sealing pits in a tooth structure in a subject in need thereof comprising contacting a tooth structure with an effective amount of one or more occlusion agents and one or more bioadhesive polymers.

### BRIEF DESCRIPTION OF THE DRAWINGS

FIG. 1 depicts the application of a composition of the invention to a glass slide, which was then weighed and then submerged in a beaker and agitated for 1 minute.

FIG. 2 illustrates the results of an in vitro conductance test as set forth herein.

FIG. 3 depicts the results of an in vitro dose response study to determine the optimal bioactive and bio-acceptable glass level for rapid occlusion of tubules.

FIG. 4 depicts the acid resistance of the two systems set forth herein, as tested in vitro.

FIG. 5 depicts the results of conductance experiments with 10% Novamin toothpaste vs. conventional non-occlusion silica toothpaste control. Confocal laser microscopy images illustrate Novamin dose response and the boosting effect of AC43 silica. The top line represents Novamin, the bottom line represents the control sample.

### DETAILED DESCRIPTION OF THE INVENTION

### General Description of the Invention

The invention encompasses oral care compositions including one or more active components, for example, one or more occlusion agents and one or more bioadhesive components including PEG/PPG copolymers, polyvinylmethyl-ether/maleic acid, cross-linked PVP, shellac, ester gum, and combinations thereof.

In certain embodiments, the active component includes an occlusion agent, anti-caries agent, a fluoride source, an agent to treat xerostomia, a desensitizing agent, and/or whitener or teeth bleach, bioactive glass, an antibacterial agent, arginine bicarbonate/calcium carbonate, and an abrasive, or combinations thereof.

In certain embodiments, the occlusion agent is bioactive glass, arginine/calcium carbonate, arginine bicarbonate/calcium carbonate, and small particle silica or combinations.

In certain embodiments, the occlusion agent comprises 1 wt. % to 50 wt. %; 5 wt. % to 40 wt. %; 10 wt. % to 30 wt. %; 15 wt. % to 20 wt. % by weight of the composition. In other embodiments, the occlusion agent comprises 50 wt. %; 40 wt. %; 30 wt. %; 20 wt. %; 10 wt. %; 5 wt. %; 4 wt. %; 3 wt. %; 2 wt. %; 1 wt. % by weight of the composition.

In certain embodiments, the bioadhesive component comprises PEG/PPG copolymers.

In certain embodiments, the bioadhesive component comprises polyvinylmethylether/maleic acid.

In certain embodiments, the bioadhesive component comprises cross-linked PVP.

In certain embodiments, the bioadhesive component includes shellac.

In certain embodiments, the bioadhesive component includes ester gum.

In certain embodiments, the bioadhesive polymer component comprises 0.1 wt. % to 70 wt. % by weight of the composition. In certain embodiments, the bioadhesive polymer component comprises 5 wt. % to 20 wt. % by weight of the composition. In certain embodiments, the bioadhesive polymer component comprises 1 wt. % to 50 wt. %; 5 wt. % to 40 wt. %; 10 wt. % to 30 wt. %; 15 wt. % to 20 wt. % by weight of the composition. In other embodiments, the bioadhesive polymer component comprises 50 wt. %; 40 wt. %; 30 wt. %; 20 wt. %; 10 wt. %; 5 wt. %; 4 wt. %; 3 wt. %; 2 wt. %; 1 wt. % by weight of the composition.

In certain embodiments, the active agent is an anti-caries agent.

In certain embodiments, the active agent is a fluoride source.

In certain embodiments, the active agent is an agent treat xerostomia.

In certain embodiments, the active agent is a desensitizing agent.

In certain embodiments, the active agent is a whitener or teeth bleach.

In certain embodiments, the active agent is bioactive glass.

In certain embodiments, the active agent is an antibacterial agent.

In certain embodiments, the active agent is arginine bicarbonate/calcium carbonate.

In certain embodiments, the active agent is an abrasive comprising silica.

In certain embodiments, the active component comprises 1 wt. % to 50 wt. %; 5 wt. % to 40 wt. %; 10 wt. % to 30 wt. %; 15 wt. % to 20 wt. % by weight of the composition. In other embodiments, the active agent comprises 50 wt. %; 40 wt. %; 30 wt. %; 20 wt. %; 10 wt. %; 5 wt. %; 4 wt. %; 3 wt. %; 2 wt. %; 1 wt. % by weight of the composition.

In another embodiment, the invention encompasses an oral care composition including an active component including an occlusion agent, anti-caries agent, a fluoride ion source, an agent treat xerostomia, an antibacterial agent, an antisensitivity agent, a tooth whitening agent, bioactive glass, an antibacterial agent, arginine bicarbonate/calcium carbonate, and particle silica or combinations thereof and one or more bioadhesive components comprising PEG/PPG copolymers, polyvinylmethylether/maleic acid, cross-linked PVP, shellac, ester gum, and combinations thereof.

In certain embodiments, the composition is a tooth varnish;

In another embodiment, the invention encompasses a method to treat a tooth comprising applying the composition of the invention to a tooth for an effective amount of time.

In certain embodiments, the composition remains on the tooth for at least 24 hours.

In certain embodiments, the composition is applied to a plurality of teeth.

In certain embodiment, the compositions are paint-on formulations, for example a varnish.

In certain embodiments, the varnish may be applied by brush, for example, dipping a brush into the composition, and then applying it to a tooth surface, for example, a dry tooth surface. In certain embodiments, the varnish is temporary, and wears off of the tooth surface after a period of time, for example, within 48 hours of application, within 24 hours of application, within 12 hours of application, within 6 hours of application, or within 2 hours of application.

Without being limited by theory, it is believed that the addition of one or more bioadhesive polymers was found to enhance in vitro efficacy and retention. The use of such compositions does not cause a reduction of activity of the active component.

### Compositions of the Invention

Throughout the disclosure, ranges are used as a shorthand for describing each and every value that is within the range. Any value within the range can be selected as the terminus of the range.

The invention encompasses oral care compositions including (i) one or more oral actives, for example, occlusion agents, fluoride ion sources, antibacterials, tooth whitening and/or bleaching agents, and antisensitivity agents, and (ii) one or more bioadhesive polymers, which facilitates adhesion of the active component to the dental surface, and to form a substantially continuous film over the surface to which the invention is applied. The bioadhesive polymer component includes PEG/PPG copolymers (e.g., BASF Pluracare L1220), polyvinylmethyl-ether/maleic acid copyolmer (e.g., Gantrez, ISP), cross-linked PVP (e.g. Polyplasdone, ISP), shellac (e.g., R49 Shellac, Mantrose-Hauser), ester gum (e.g. Eastman Chemicals), and combinations thereof.

### Polymer Bioadhesive Agents

The bioadhesive polymer may include any polymer that promotes adhesion of the active agent to teeth. In certain embodiments, the polymer bioadhesive may become more adhesive when the adhesive composition or layer is moistened with, for example, water or saliva.

The term “bioadhesive polymer” is broadly defined as a polymer that allows continued contact of an active ingredient with the teeth or a tooth surface and retained on the teeth or tooth surface for an extended period of time, for example, 1 hour, 3 hours, 5 hours, 10 hours, 24 hours. In certain embodiments, the “bioadhesive polymer” is a polymer that is capable of being bound to the teeth or a tooth surface to allow continued contact of an active ingredient to the teeth or tooth surface. In other embodiments, the bioadhesive polymer is a material or combination of materials that enhance the retention of the active ingredient on the teeth or a tooth surface onto which the composition is applied. Such bioadhesive polymers include, for example, hydrophilic organic polymers, hydrophobic organic polymers, silicone gums, silicas, and combinations thereof.

In certain embodiments, the bioadhesive agent comprises a bioadhesive polymer selected from the group consisting of PEG/PPG copolymers, polyvinylmethylether/maleic anhydride copolymers, polyvinylpyrrolidone, cross-linked PVP, shellac, polyethylene oxide, methacrylates, acrylates copolymers, methacrylic copolymers, vinylpyrrolidone/vinyl acetate copolymers, polyvinyl caprolactum, polylactides, silicone resins, silicone adhesives, chitosan, milk proteins (casein), amelogenin, ester gum, and combinations thereof.

In various embodiments, the bioadhesive polymer includes, but is not limited to, PEG/PPG copolymers (e.g., BASF Pluracare L1220), polyvinylmethyl-ether/maleic acid copyolmer (e.g., Gantrez, ISP), cross-linked PVP (e.g., Polyplasdone, ISP), shellac (e.g., R49 Shellac, Mantrose-Hauser), ester gum (e.g., Eastman Chemicals), and combinations thereof.

In certain embodiments, the bioadhesive polymer is polyvinyl pyrrolidone (PVP). PVP polymers have been found to provide superior adhesion to teeth when a surface of a substantially solid adhesive composition is moistened with saliva or water.

In various embodiments, the bioadhesive polymer includes a hydrophilic organic polymers including, but not limited to, polyethylene glycols, nonionic polymers of ethylene oxide, block copolymers of ethylene oxide and propylene oxide, carboxymethylene polymers, polyvinyl pyrrolidone (PVP) and mixtures thereof. Nonaqueous hydrophilic polymers useful in the practice of the present invention in certain embodiments provide a viscosity for the composition in the amount of 10,000 mPas (cps) to 600,000 mPas (cps).

In other embodiment, the bioadhesive polymer includes hydrophilic polymers including polymers of polyethylene glycols and ethylene oxide having the general formula: HOCH₂(CH₂OCH₂)ₙOH, wherein n represents the average number of oxyethylene groups. Polyethylene glycols available from Dow Chemical (Midland, Mich.) are designated by number such as 200, 300, 400, 600, 2000 which represents the approximate weight average molecular weight of the polymer. Polyethylene glycols 200, 300, 400, and 600 are clear viscous liquids at room temperature, and are used in certain embodiments of the present invention.

In other embodiment, the bioadhesive polymer includes water soluble, nonionic block copolymer of ethylene oxide and propylene oxide of the formula:

HO(C₂H₄O)ₐ(C₃H₆O)_{b}(C₂H₄O)CH.

The block copolymer in certain embodiments is chosen (with respect to a, b and c) such that the ethylene oxide constituent comprises 65 to 75% by weight, of the copolymer molecule and the copolymer has a weight average molecular weight of 2,000 to 15,000, with the copolymer being present in oral care composition in such concentration that the composition is liquid at room temperature (23° C.).

In other embodiment, the bioadhesive polymer includes PLURAFLO™ L1220 of BASF Corporation, which has a weight average molecular weight of 9,800. The hydrophilic poly(ethylene oxide) block averages 65% by weight of the polymer.

In other embodiment, the bioadhesive polymer includes organic polymers useful as adhesion enhancing agents include hydrophilic polymers such as carbomers such as carboxymethylene polymers such as acrylic acid polymers, and acrylic acid copolymers. Carboxypolymethylene is a slightly acidic vinyl polymer with active carboxyl groups. A carboxypolymethylene is CARBOPOL™ 974 marketed by Noveon, Inc., Cleveland, Ohio, U.S.A.

In other embodiment, the bioadhesive polymer includes hydrophobic organic materials including polyethylene blends, petrolatum, white petrolatum, liquid paraffin, butane/ethylene/styrene hydrogenated copolymer) blends (VERSAGEL™ marketed by Penreco, Houston, Tex., U.S.A.), acrylate and vinyl acetate polymers and copolymers, polyethylene waxes, silicone polymers as discussed further herein and polyvinyl pyrrolidone/vinyl acetate copolymers. In embodiments of the present invention containing a hydrophobic polymer, they can be present in amounts of 1 to 85% weight of the composition.

In other embodiment, the bioadhesive polymer includes inorganic materials for example silicon polymers such as amorphous silica compounds which function as thickening agents (CAB-O-SIL™ fumed silica manufactured by Cabot Corporation, Boston, Mass., U.S.A.; and SYLOX™ 15 also known as SYLODENT™ 15, marketed by Davison Chemical Division of W.R. Grace & Co., Columbia, Md., U.S.A.).

In other embodiments, polymers may include one or more of acrylate copolymers (such as terpolymer of t-butyl acrylate, ethyl acrylate, and methacrylic acid, BASF Luvimer Pro55; or copolymer of acrylic acid, methyl acrylate, 2-acrylamido-2-methylpropanesulfonic acid, BASF Lupasol FF4243), vinylpyrrolidone/vinyl acetate copolymer (such as BASF Luviskol VA 37E), methacrylic copolymers (such as Evonik Eudragit), polyethylene oxide (such as Dow Polyox (PEG2M)), and polyvinylmethylether/maleic anhydride copolymers (ISP Gantrez).

In other embodiments, the bioadhesive polymer includes a lac material. Lac is a natural resinous substance excreted by an insect, Laccifer Lacca, and has been used in dentistry. (See A. Azucca, R. Huggett, and A. Harrison, “The Production of Shellac and its General and Dental Uses: A review.” Journal of Oral Rehabilitation, 1993, vol. 20, pp. 393 400; and I. Klineberg and R. Earnshaw, “Physical Properties of Shellac Baseplate Materials.” Australian Dental Journal, October, 1967, vol. 12 no. 5, pp. 468 475.) Another use of shellac in dentistry includes treatment of a cavity with a hydrophilic shellac film placement of a polystyrene liner. (See M. Blixt and P. Coli, “The Influence of Lining Techniques on the Marginal Seals of Class II Composite Resin Restorations” Quintessence International, vol. 24, no. 3, 1993). Shellac has also been prepared and used in dentistry for the use of a bead adhesive for securing a composite resin veneer cast restoration. (See, e.g., C. Lee, H. Pierpont, and E. Strickler, “The Effect of Bead Attachment Systems on Casting Patterns and Resultant Tensile Bond Strength of Composite Resin Veneer Cast Restorations,” The Journal of Prosthetic Dentistry, November, 1991, vol. 66, no. 5, pp. 623 630). In various embodiments, the shellac or lac compositions of the invention are non-toxic and may be used to incorporate glass microspheres to produce a temporary cosmetic dental coating.

In other embodiments, the polymer adhesion agent includes a shellac; in certain embodiments, the shellac is a dewaxed bleached shellac. Without being limited by theory, it is believed that a bleached shellac imparts less color when applied to a tooth, and has greater stability, for example, the phases tend not to separate.

In other embodiments, the composition includes bleached shellac in an amount of 5% to 70% weight of the composition, e.g., from 5% to 40%, from 10% to 30%, or 20%, or wherein the bleached shellac comprises from 10% to 50% by weight of the adhesive film forming component, e.g., from 15% to 35%, or 25% by weight of the component.

### Inert Components

The bioadhesive compositions may include inert components in addition to the polymer bioadhesion agent to yield a final composition or layer having desired properties. Examples of “inert” components include, but are not limited to, plasticizers and humectants (e.g., glycerin, sorbitol, polyethylene glycol, propylene glycol, and polypropylene glycol), volatile solvents (e.g., water and alcohols, such as ethanol), stabilizing agents (e.g., EDTA and citric acid), neutralizing agents. (e.g., sodium hydroxide), thickening agents (e.g., fumed silica), flavorants, sweeteners, and the like.

When water is used as a solvent when manufacturing adhesive compositions or layers according to the invention and then driven off by evaporation to yield a substantially solid dental bleaching or desensitizing composition, it is postulated that a significant amount of water remains bound or associated with the hydrophilic components within the adhesive composition, including the tooth adhesion agent, any inert components (e.g., polyols added as humectants, stabilizing agents, neutralizing agents, and/or thickening agents), and any hydrophilic active agents (e.g., bleaching and/or desensitizing agents). Although the amount of residual water has not yet been determined, it is believed that approximately 10% of the water added initially remains after the initially flowable adhesive composition intermediate has been dried sufficiently to yield the substantially solid adhesive composition or layer.

### Active Agents

The compositions of the invention include one or more active component including an occlusion agent, anti-caries agent, a fluoride source, an agent treat xerostomia, a desensitizing agent, and/or whitener or teeth bleach, bioactive glass, an antibacterial agent, arginine bicarbonate/calcium carbonate, and an abrasive, or combinations thereof.

1. Occlusion Agents

Occlusion agents of the invention include, but are not limited to, bioactive glass, arginine/calcium carbonate, arginine bicarbonate/calcium carbonate, and small particle silica or combinations. As used herein, the term “occlusion agent” refers to any agent that aids in remineralization of the teeth or a tooth surface or agents that deposit compounds on and in the tooth surface and when applied to dental tissue prevent and/or repair dental weaknesses. For example, bioactive glass such as amorphous calcium compounds including amorphous calcium phosphate, amorphous calcium phosphate fluoride and amorphous calcium carbonate phosphate for use in remineralizing teeth. The occlusion agents of the invention when applied to dental tissue prevent and/or repair dental weaknesses

A. Bioactive Glasses

The compositions of the invention generally include one or more bio-acceptable, bioactive glasses.

Suitable bioacceptable and bioactive glasses for use in the invention include, but are not limited to, an inorganic glass material capable of forming a layer of hydroxycarbonate apatite in accordance with the present invention. In one embodiment, the dentifrice composition of the present invention includes a bioactive and bioacceptable glass. In one embodiment, the composition includes calcium sodium phosphosilicate. In one embodiment, the composition includes calcium sodium phosphosilicate in an amount from 1.0 wt. % to 20 wt. %. In one embodiment, the composition includes calcium sodium phosphosilicate in an amount from 5.0 wt. % to 15 wt. %. In one embodiment, the composition includes calcium sodium phosphosilicate in an amount of 10 wt. %.

Suitable bioacceptable and bioactive glasses may have compositions including: from 40 wt. % to 86 wt. % of silicon dioxide (SiO₂); from 0 wt. % to 35 wt. % of sodium oxide (Na₂O); from 4 wt. % to 46 wt. % of calcium oxide (CaO); and from 1 wt. % to 15 wt. % of phosphorus oxide (P₂O₅). Preferably, the bioacceptable and bioactive glass includes: from 40 wt. % to 60 wt. % of silicon dioxide (SiO₂); from 10 wt. % to 30 wt. % of sodium oxide (Na₂O); from 10 wt. % to 30 wt. % of calcium oxide (CaO); and from 2 wt. % to 8 wt. % of phosphorus oxide (P₂O₅). The oxides may be present as solid solutions or mixed oxides, or as mixtures of oxides. Exemplary bioacceptable and bioactive glass suitable for use in the present invention include NovaMin®, which has a composition including 45 wt. % of silicon dioxide, 24.5 wt. % of sodium oxide, 6 wt. % of phosphorus oxide, and 24.5 wt. % of calcium oxide.

In one embodiment, the composition of suitable bioacceptable and bioactive glass may also include: CaF₂, B₂O₃, Al₂O₃, MgO and K₂O, in addition to silicon, sodium, phosphorus and calcium oxides. In certain embodiments, the range of CaF₂ is from 0 wt. % to 25 wt. %. The preferred range for B₂O₃ is from 0 wt. % to 10 wt. %. The preferred range for Al₂O₃ is from 0 wt. % to 4 wt. %. The preferred range for MgO is from 0 wt. % to 5 wt. %. The preferred range for K₂O is from 0 wt. % to 8 wt. %.

An “effective” amount of the bio-acceptable and bioactive glass is an amount that is sufficient to have the desired therapeutic or prophylactic effect in the human or lower animal subject to whom the active is administered, without undue adverse side effects (such as toxicity, irritation, or allergic response), commensurate with a reasonable benefit/risk ratio when used in the manner of this invention. The specific effective amount will vary with such factors as the particular condition being treated, the physical condition of the subject, the nature of concurrent therapy (if any), the specific active used, the specific dosage form, the carrier employed, and the desired dosage regimen.

The bioactive glasses of the invention provide an efficacious material for interaction with the tooth structure. A biocompatible glass in accordance with the invention is one that does not trigger an adverse immune response.

In accordance with the invention, it has been found that bioactive glasses of specified particle sizes are particularly useful in treating the above-mentioned conditions. Specifically, surprising results are obtained by the compositions of the invention where small and very small particles are combined. In certain embodiments, for example, the bioactive glass portion of the compositions include small particles that are capable of bonding with tooth structure (e.g., less than 90 microns) as well smaller particles (e.g., less than 10) are used in combination, the larger of these particles adhere to tooth structure and act as ionic reservoirs while the smaller are capable of entering and lodging inside of various tooth structure surface irregularities.

In one embodiment, bioacceptable and bioactive glass suitable for use in the present invention is particulate, non-interlinked bioactive glass. In one embodiment, the glass has a particle size range of less than 90 μm. In one embodiment, the glass has a particle size range of less than 70 μm. In one embodiment, the glass has a particle size range of less than 50 μm. In one embodiment, the glass has a particle size range of less than 40 μm. In one embodiment, the glass has a particle size range of less than 30 μm. In one embodiment, the glass has a particle size range of less than 20 μm. In certain embodiments, the particle size of the bioactive glass portion of the compositions is less than 20, 10, 5, 4, 3, 2, 1 micron.

In an embodiment, a glass has a median particle size between 0.5 μm and 90 μm. In another embodiment, a glass has median a particle size between 0.5 μm and 70 μm. In another embodiment, a glass has a median particle size between 0.5 μm and 50 μm. In another embodiment, a glass has a median particle size between 0.5 μm and 40 μm. In another embodiment, a glass has a median particle size between 0.5 μm and 30 μm. In another embodiment, a glass has a median particle size between 0.5 μm and 20 μm. In another embodiment, a glass has a median particle size between 0.5 μm and 10 μm. In another embodiment, a glass has a median particle size between 0.5 μm and 5 μm. In another embodiment, a glass has a median particle size between 0.5 μm and 4 μm. In another embodiment, a glass has a median particle size between 0.5 μm and 3 μm. In another embodiment, a glass has a median particle size between 0.5 μm and 2 μm. In another embodiment, a glass has a median particle size between 0.5 μm and 1 μm. In yet another embodiment, a glass has a median particle size selected from the group consisting of 0.5 μm, 1 μm, 2 μm, 3 μm, 4 μm, 5 μm, 7.5 μm and 10 μm.

In certain embodiments, the larger of these particles (e.g., less than 90 microns to less than 20 microns) provide a reservoir of additional calcium and phosphorous so that the mineralization, or depositing of the calcium phosphate layer begun by the small particles (e.g., less than 20 microns to less than 1 micron) can continue. In certain embodiments of the invention, additional calcium and phosphorous can be leached to all tooth structure as well as to particles, which have become attached to the inside or at the openings of surface irregularities of tooth structure such as dentinal tubules. This in turn provides for continuation of the entire reaction and continued growth of the smaller of these particles, which have lodged inside or over the openings of such surface irregularities and can result in effectively coating or filling the surface irregularity. This excess concentration of ions of calcium and phosphorous allows reaction of the smaller of these particles to take place because the smaller particles quickly exhaust their ions because of their relatively high surface area. The larger of these particles will react and release their ions more slowly as a longer term effect. Furthermore, the larger of these particles will mechanically abrade the tooth surface opening various surface irregularities allowing small particles to enter and react with the surface irregularity.

This effect is very beneficial in a variety of applications. For example, in preventing caries or decay, the compositions of the invention are capable of penetrating into the depths of the smallest of surface irregularities and receiving a continued supply of ions from larger nearby particles so that it is able to grow after exhausting its stored ion supply. This is also very useful in sealing pits and fissures, and a much more effective and long lasting seal is obtained.

The occlusion of these tubules leads to a significant reduction in the amount of sensitivity after, for example, periodontal surgery. In certain embodiments, a mixture of particles less than two microns and larger than 45 microns in diameter are used. It has been found that this combination yields a particularly effective composition.

In certain embodiments, the bio-acceptable and bioactive glass encompasses glass compositions including the following components by weight:

| Ingred. | wt. % |
|---|---|
| SiO₂ | 40-60 |
| CaO₂ | 10-30 |
| Na₂O | 10-35 |
| P₂O₅ | 2-8 |
| CaF₂ | 0-25 |
| B₂O₃ | 0-10 |

In certain embodiments, the following composition by weight percentage encompasses a bioactive glass:

| Ingred. | wt. % |
|---|---|
| SiO₂ | 40-60 |
| CaO₂ | 10-30 |
| Na₂O | 10-35 |
| P₂O₅ | 2-8 |
| CaF₂ | 0-25 |
| B₂O₃ | 0-10 |
| K₂O | 0-8 |
| MgO | 0-5 |

In various embodiments, the bioactive glass is present in the compositions in an amount of 1 wt. % to 35 wt. %, 5 wt. % to 30 wt. %, 10 wt. % to 25 wt. %, 15 wt. % to 20 wt. %, and 20 wt. %.

B. Arginine Bicarbonate/Calcium Carbonate

In certain embodiments, the occlusion agent includes an arginine bicarbonate, an amino acid complex, and particles of calcium carbonate. In certain embodiments, arginine bicarbonate/calcium carbonate is an abrasive. In certain embodiments, the arginine bicarbonate/calcium carbonate complex creates an alkaline environment to further enhance particle attachment.

In certain embodiments, the arginine-bicarbonate/calcium carbonate compositions can counter tooth mineral loss in dental caries and dentinal hypersensitivity. In other embodiments, the These arginine-bicarbonate/calcium carbonate compositions are capable of neutralizing acid production and remineralizing tooth structure.

In various embodiments, the arginine bicarbonate/calcium carbonate is present in the compositions in an amount of 1 wt. % to 35 wt. %, 5 wt. % to 30 wt. %, 10 wt. % to 25 wt. %, 15 wt. % to 20 wt. %, and 20 wt. %.

C. Small Particle Silica

In certain embodiments, the occlusion agent includes silica, in certain embodiments small particle silica. A composite restorative material which is widely used in the dental field in recent years is required to have the following properties. In certain embodiments, the small particle silica includes an ultrafine particle having an average particle size of 0.01 μm to 100 μm, 0.1 μm to 50 μm, 1 μm to 10 μM, and 5 μm, or combinations thereof.

In an aspect, suitable silica particles may have, for example, a median particle size of 8 microns or less, alternatively, a median particle size of 3 to 4 microns, alternatively, a median particle size of 5 to 7 microns, alternatively, a median particle size of 3 to 5 microns, alternatively, a median particle size of 2 to 5 microns, or alternatively, a median particle size of 2 to 4 microns.

In another aspect, the oral compositions within the scope of the invention also include particles that have a median particle size that is no greater than the average diameter of a mammalian dentin tubule, such that one or more particles is/are capable of becoming lodged within the tubule, thereby effecting a reduction or elimination of perceived tooth sensitivity.

In addition, the presence of small particle silica acted as a pH buffer to bring the formulation into the pH range accepted by ISO standards as well as offering addition occlusion benefit. In vitro retention and dentin conductance studies showed significant improvement in retention, reduction in dentinal fluid flow, and acid resistance when compared to the previously consumer and clinically tested control product

In various embodiments, the small particle silica is present in the compositions in an amount of 1 wt. % to 35 wt. %, 5 wt. % to 30 wt. %, 10 wt. % to 25 wt. %, 15 wt. % to 20 wt. %, and 20 wt. %.

2. Other Active Agents

A. Tartar Control Agent

In some embodiments, compositions of the invention may optionally comprise an additional active agent including, but not limited to, a tartar control (anti-calculus) agent formulated to not interfere with the efficacy of the bioactive glass and/or potassium salts described in detail herein. Tartar control agents among those useful herein include salts of any of these agents, for example their alkali metal and ammonium salts: phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclo-pentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof

B. Fluoride Sources

Fluoride sources suitable for use in the present invention may include any orally acceptable particulated fluoride-ion containing agent formulated to not interfere with the efficacy of the bioactive glass, and that may be useful, for example, as an anti-caries agent. Suitable fluoride sources may include, but are not limited to: ionic fluorides including alkali metal fluorides; amine fluorides such as olaflur (N′-octadecyltrimethylendiamine-N,N,N-tris(2-ethanol)-dihydrofluoride), indium fluoride, sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluoride, or combinations thereof; and ionic monofluorophosphates including alkali metal monofluorophosphates such as potassium, sodium and ammonium fluorides and monofluorophosphates; and mixtures thereof.

In one embodiment, a dentifrice composition of the present invention further includes a fluorine source. In one embodiment, a composition further includes a fluoride salt. In one embodiment, a composition further including a fluoride salt includes sodium monofluorophosphate. In one embodiment, calcium glycerophosphate, which has been shown to enhance the activity of ionic monofluorophosphates, may be optionally added when the fluoride source is an ionic monofluorophosphate. In one embodiment, a composition may include a fluorine source providing between 100 and 3000 ppm of fluoride. In one embodiment, a composition may include a fluorine source providing between 500 and 2000 ppm of fluoride.

C. Whitening Agents

Whitening agents suitable for use in the present invention may include any therapeutically effective agent suitable for use in an oral cavity. Suitable whitening agents include, but are not limited to: titanium dioxide, hydrogen peroxide, sodium tripolyphosphate, and the like. In one embodiment, a dentifrice composition of the present invention further includes a whitening agent. In one embodiment, a composition of the invention further includes titanium dioxide. In one embodiment, titanium dioxide may be included at appropriate levels.

D. Abrasives

Suitable abrasives for use in the present invention may include, but are not limited to: silica, zinc orthophosphate, sodium bicarbonate (baking soda), plastic particles, alumina, hydrated alumina, calcium carbonate, calcium pyrophosphate, and mixtures thereof. The silica abrasive may be a natural amorphous silica including diatomaceous earth; or a synthetic amorphous silica such as a precipitated silica; or a silica gel, such as a silica xerogel; or mixtures thereof.

Generally, an amount of abrasive suitable for use in the dentifrice composition of the invention will be empirically determined to provide an acceptable level of cleaning and polishing, in accordance with the techniques well known in the art. In one embodiment, a dentifrice composition of the present invention includes an abrasive. In one embodiment, a composition includes a silica abrasive. In one embodiment, a silica abrasive is present in an amount of from 1 wt. % to 30 wt. %. In one embodiment, a silica abrasive is present in an amount of from 5 wt. % to 15 wt. %. In one embodiment, a silica abrasive is present in an amount of from 7 wt. % to 10 wt. %.

E. Mouth-Feel Agents

Mouth-feel agents suitable for use in the present invention may include any orally acceptable materials imparting a desirable texture or other feeling during use of the dentifrice composition, in any form or amount. Suitable mouth-feel agents may include, but are not limited to: dispersed flavorants, sweeteners, saliva-stimulating agents, and the like.

Flavorants among those useful herein include any material or mixture of materials operable to enhance the taste of the composition. Any orally acceptable natural or synthetic flavorant can be used, such as flavoring oils, flavoring aldehydes, esters, alcohols, similar materials, and combinations thereof. Flavorants include vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants, and mixtures thereof. Also encompassed within flavorants herein are ingredients that provide fragrance and/or other sensory effect in the mouth, including cooling or warming effects. Such ingredients include menthol, menthyl acetate, menthyl lactate, camphor, eucalyptus oil, eucalyptol, anethole, eugenol, cassia, oxanone, alpha-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA), and mixtures thereof. One or more flavorants are optionally present in a total amount of 0.01% to 5%, optionally in various embodiments from 0.05 to 2%, from 0.1% to 2.5%, and from 0.1 to 0.5%.

Sweeteners among those useful herein include orally acceptable natural or artificial, nutritive or non-nutritive sweeteners. Such sweeteners include dextrose, polydextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, sucralose, dipeptide-based intense sweeteners, cyclamates, dihydrochalcones, and mixtures thereof. One or more sweeteners are optionally present in a total amount depending strongly on the particular sweetener(s) selected, but typically at levels of from 0.005% to 5%, optionally from 0.01% to %.

The compositions of the present invention may optionally comprise a saliva stimulating agent formulated to not interfere with the efficacy of the bioactive glass and/or potassium salts described in detail herein and useful, for example, in amelioration of dry mouth. One or more saliva stimulating agents are optionally present in saliva stimulating effective total amount.

F. Other Active Ingredients

In some embodiments, compositions of the invention may optionally include other active materials, operable for the prevention or treatment of a condition or disorder of hard or soft tissue of the oral cavity, or the prevention or treatment of a physiological disorder or condition. In some embodiments, the active is a “systemic active” which is operable to treat or prevent a disorder that, in whole or in part, is not a disorder of the oral cavity. In some embodiments, the active is an “oral care active” operable to treat or prevent a disorder or provide a cosmetic benefit within the oral cavity (e.g., to the teeth, gingiva or other hard or soft tissue of the oral cavity). Oral care actives among those useful herein include whitening agents, anticaries agents, tartar control agents, antiplaque agents, periodontal actives, abrasives, breath freshening agents, tooth desensitizers, salivary stimulants, and combinations thereof.

In some embodiments, compositions of the invention may optionally include an antibacterial agent formulated to not interfere with the efficacy of the bioactive glass and/or potassium salts described in detail herein. Examples of antibacterial agents include, but are not limited to, triclosan, cetylpyridinium chloride, and combinations thereof.

In some embodiments, compositions of the invention include comprise a nutrient formulated to not interfere with the efficacy of the bioactive glass and/or potassium salts described in detail herein. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), Gantrez, amelogenin, milk proteins (casein), chitosan, pluracare L1220 (ethylene oxide/propylene oxide copolymer), polyox, PVP, methacrylates, shellac, arginine, and mixtures thereof.

In some embodiments, compositions of the invention may also contain an antistain agent. Suitable antistain agents may include, but are not limited to: carboxylic acids, amino carboxylate compounds, phosphonoacetic acid, polyvinylpyrrolidone, and the like. The antistain agent may be incorporated into the dentifrice composition or may be provided as a separate composition, for use after the dentifrice.

In some embodiments, compositions of the invention may also contain beeswax, colophonium, mastic, a water-insoluble alkyl cellulose, and combinations thereof.

In some embodiments, compositions of the present invention may also contain a solvent, for example, wherein the composition comprises from 5% to 50% weight of the solvent, e.g., from 10% to 40%, from 25% to 30%, or 27%.

In some embodiments, the solvent is selected from methanol, ethanol, ethyl acetate, acetone, isopropyl alcohol, or combinations thereof;

In some embodiments, compositions of the invention may also contain a tooth desensitizing agent comprising a tooth desensitizing agent selected from a potassium salt, capsaicin, eugenol, a strontium salt, a zinc salt, a chloride salt, or combinations thereof;

In some embodiments, compositions of the invention may also contain a stannous ion agent, triclosan, triclosan monophosphate, chlorhexidine, alexidine, hexetidine, sanguinarine, benzalkonium chloride, salicylanilide, arginate esters, ethyl lauryl arginate, bisphenols, domiphen bromide, tetradecylpyridinium chloride, N-tetradecyl-4-ethylpyridinium chloride, octenidine, delmopinol, octapinol, nisin, zinc ion agent, copper ion agent, essential oils, furanones, bacteriocins, a basic amino acid, or combinations thereof.

### Methods of Treating and Preventing Disorders of the Oral Cavity

The oral care compositions of the invention include, in part, one or more active agents and one or more bioadhesive polymer components that are useful in treating or preventing various disorders of the oral cavity in a subject in need thereof, for example, enamel remineralization, incipient caries remineralization, carious dentin remineralization, caries prevention, arresting decay, reversing decay, anti-caries, pit and fissure sealants, prophylactic pastes, fluoride treatments, dentinal sealants, and combinations thereof. As used herein, the term “subject” includes mammals, for example, humans and companion animals including cats and dogs.

Additional methods of treating or preventing disorders of the oral cavity are also included within the scope of the invention. In one embodiment, a method of at least partially occluding dentin tubules in a subject in need thereof includes contacting the teeth or a tooth surface with an oral care composition in accordance with the invention. In one embodiment, a method of preventing tooth decay in a subject in need thereof includes contacting the teeth or a tooth surface with an oral care ntifrice composition in accordance with the invention. In one embodiment, a method of treating tooth decay in a subject in need thereof includes contacting the teeth or a tooth surface with an oral care composition in accordance with the invention. In one embodiment, a method of preventing incipient carries in a subject in need thereof includes contacting the teeth or a tooth surface with an oral care composition in accordance with the invention. In one embodiment, a method of remineralizing enamel in a subject in need thereof includes contacting the teeth or a tooth surface with an oral care composition in accordance with the invention. In one embodiment, a method of sealing fissures in a subject in need thereof includes contacting the teeth or a tooth surface with an oral care composition in accordance with the invention. In one embodiment, a method of sealing pits in a subject in need thereof includes contacting the teeth or a tooth surface with an oral care composition in accordance with the invention. In one embodiment, a method of lining tooth structure in a subject in need thereof includes contacting the teeth or a tooth surface with an oral care composition in accordance with the invention. In one embodiment, a method for capping pulp in a subject in need thereof includes contacting the teeth or a tooth surface with an oral care composition in accordance with the invention. In one embodiment, a method for treating tooth structure after periodontal surgery in a subject in need thereof includes contacting the teeth or a tooth surface with an oral care composition in accordance with the invention.

### EXAMPLES

The invention will now be described with respect to the following non-limiting examples:

### Example 1

Suitable bioadhesive polymers include PEG/PPG copolymers (BASF Pluracare L1220), polyvinylmethylether/maleic acid copyolmer (Gantrez, ISP), cross-linked PVP (Polyplasdone, ISP), shellac (R49 Shellac, Mantrose-Hauser), and ester gum (Eastman Chemicals). The occlusion agent for desentization that can be used in the formulas includes bioactive glass, arginine/calcium carbonate, arginine bicarbonate/calcium carbonate (Cavistat/PCC), and small particle silica or combinations of these.

An example of a suitable small particle silica includes Sorbosil AC43 from Ineos. Table 1 below illustrates an illustrative example of a composition with bioactive glass and resulting pH.

TABLE 1

| | Control | A | B | C | D |
|---|---|---|---|---|---|
| Ingredient | wt % | wt % | wt % | wt % | wt % |
| Glycerin | 25 | | | | 25 |
| Pluracare L1220 | | 40 | 25 | 25 | |
| Pluracare L4370 | | 28.7 | 28.7 | 26.7 | |
| Silicone Fluid | | 20 | 20 | 20 | |
| Bioactive Glass | 5 | 10 | 10 | 10 | 10 |
| Small Particle Silica | | | 15 | 15 | 15 |
| 95% Ethyl alcohol | 27.5 | | | | 27.7 |
| Propylene Glycol | 10 | | | | |
| PEG 400 | 20 | | | | |
| Hydroxypropylcellulose | 0.2 | | | | |
| Crospovidone NF | 12 | | | | |
| Gantrez | | | | 2 | |
| Shellac | | | | | 20 |
| Saccharin, Flavor | QS | QS | QS | QS | QS |
| Total | 100 | 100 | 100 | 100 | 100 |
| 25% pH (ISO) | 11.47 | 11.90 | 10.39 | 10.19 | 9.20 |

Formula A is an example with a PEG/PPG copolymer for increased retention. Addition of small particle silica decreased the pH significantly (Formula B) into the accepted ISO range (<10.5). Small particle silica has the added benefit of also providing dentinal occlusion and increased acid resistance. Formulas C and D are examples with Gantrez and shellac.

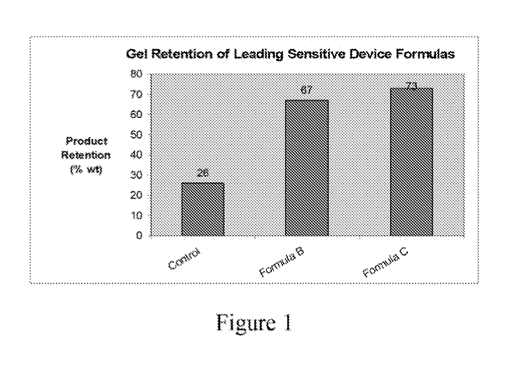

A laboratory method was developed to screen formulations for retention. An illustrative composition of the invention was applied to a glass slide, weighed and then submerged in a beaker with agitation for 1 minute. The slide was removed, dried and weighed to calculate % product retained. FIG. 1 illustrates retention of Formulas B and C with the Control. Both formulas B and C showed increased retention relative to the control formula.

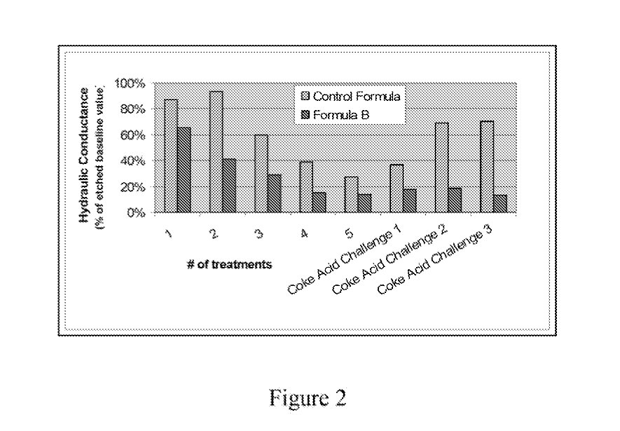

To predict efficacy, dentin conductance, a laboratory test to measure fluid flow rate through dentin segments, was measured for etched dentin treated with either Formula B or Control. After each treatment, the fluid flow rate was immediately measured using a Flodec instrument. The dentin conductance was reported as a % vs. the baseline etched value for the dentin segment. The lower the % conductance, the more occluded the dentin tubules. After the treatment phase, the segments were then soaked in Coca Cola for 1 minute to simulate an acid challenge. The fluid flow rate was measured again. FIG. 2 illustrates the results of the in vitro conductance testing.

In an embodiment, the reduction in sensitivity of a tooth is demonstrated herein, and in U.S. Patent Application Publication No. 2009/0092562, incorporated in its entirety herein by reference, by a reduction in the measured fluid flow rate, a measure of conductance of dentin. In one method, extracted human molars are cut at the crown and roots using a diamond saw. The pulp is removed and the resulting dentin segment is stably mounted, such as onto an acrylic block. Tubing is connected from a hole in the acrylic block mounting just below the pulp chamber. The dentin segment is connected to an apparatus that measures the rate of fluid flow (hydraulic conductance). See, Zhang et al., “The effects of pain free desensitizer on dentine permeability and tubule occlusion over time, in vitro”, Journal of Clinical Periodontol, 25(11 Pt 1): 884-91 (Nov. 1998), the contents of which are incorporated herein by reference.

The top surface of the dentin is etched with citric acid. The fluid flow rate across the etched dentin is measured under 70 cm water pressure. The dentin surface is then treated with a slurry of the oral composition of the invention diluted with 3 parts deionized water and the fluid flow rate is measured again. See Pashley et al., “Effects of desensitizing dentifrices in vitro,” J. Periodontol., 55 (9): 522-525 (Sep. 1984).

Formula B showed lower fluid flow rates than the Control, reaching 14% of the etched value after the fourth application. In addition, Formula B showed better acid resistance than the Control in the cola treatment phase. Thus, Formula B with the retentive polymer and small particle silica showed a significant improvement over the clinically tested Control formula in terms of both product retention and in vitro efficacy.

In addition to potential anti-sensitivity benefits from potassium salts, the potassium unexpectedly helps thicken the non-aqueous bioactive glass formula. Below is a comparison of illustrative embodiments of compositions and viscosities of formulas prepared with and without potassium chloride. Formula A with 3.7% potassium chloride shows acceptable viscosity. However, with the potassium chloride is removed from the formula (Formula B), the viscosity drops dramatically and is unacceptable. In addition increasing the silica thickener does not improve the viscosity (Formula C).

TABLE 2
Non-aqueous Toothpaste with Bioactive Glass

| Ingredients | Formula A | Formula B | Formula C |
|---|---|---|---|
| Glycerin | 58.8 | 62.6 | 55.6 |
| Bioactive Glass (Novamin ®) | 5 | 5 | 5 |
| Pluracare L1220 | 5 | 5 | 5 |
| Saccharin | 0.3 | 0.3 | 0.3 |
| Zeodent 115 Silica | 20 | 20 | 20 |
| Zeodent 165 Silica | 3 | 3 | 10 |
| (thickener) | | | |
| KCl | 3.7 | 0 | 0 |
| MFP | 1.1 | 1.1 | 1.1 |
| SLS Powder | 1.2 | 1.2 | 1.2 |
| Titanium Dioxide | 1 | 1 | 1 |
| Flavor | 0.8 | 0.8 | 0.8 |
| Total (wt. %) | 100 | 100 | 100 |
| Brookfield Viscosity | 26 | 4 | 6 |
| (>1 wk) | | | |

### Example 2

### Single-tube Toothpaste Product Including Occlusion Agent(s) and Potassium salt(s) that Offers Superior Tooth Sensitivity Relief

An illustrative embodiment of the invention encompasses a single tube toothpaste product including one or more inclusion agents and one or more potassium salts. In one illustrative embodiment, to deliver faster relief, a single tube technology that combines rapid occlusion agents, for example, a bioactive and bio-acceptable glass (e.g., Novamin) with potassium is made. The non-aqueous bioactive and bio-acceptable glass formulations with potassium were found to provide significant in vitro occlusion.

In another illustrative embodiment, the bioactive and bio-acceptable glass (e.g., Novamin) formula is surprising found to possess additional occlusion benefit by adding commercially available small particle silica (e.g., Sorbosil AC-43).

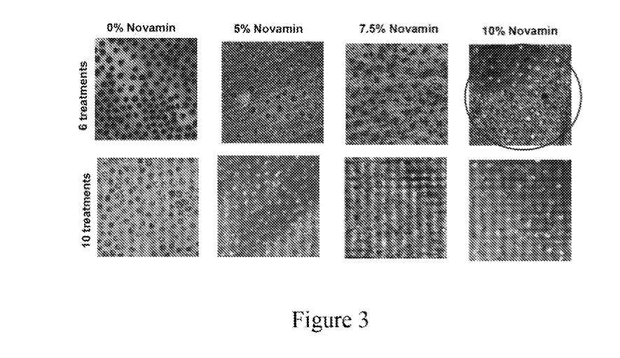

FIG. 3 illustrates an in vitro dose response study that was performed to determine the optimal bioactive and bio-acceptable glass (e.g., Novamin) level for rapid occlusion. Products with the bioactive and bio-acceptable glass (e.g., Novamin) at 5%, 7.5% and 10% were prepared. Products were evaluated by confocal microscopy after 6 and 10 brushings. After six treatments, the 10% bioactive and bio-acceptable glass (e.g., Novamin) formula showed significant occlusion while all bioactive and bio-acceptable glass (e.g., Novamin) levels provided significant occlusion after 10 treatments.

To boost the 5% bioactive and bio-acceptable glass (e.g. Novamin) occlusion at six treatments, the effect of addition of silica (e.g., Ineos AC43 silica) was studied in vitro. As shown in the confocal microscopy images below, the addition of 9% silica (e.g. Ineos AC43 silica) significantly improved occlusion at six treatments.

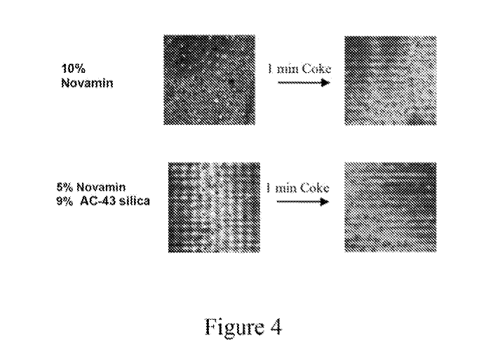

The acid resistance of the two leading systems was evaluated in vitro (FIG. 4). The 6-treatment dentin disks were soaked for 1 minute in Coke Classic. Images are shown below. Both systems showed significant resistance to acid challenge.

To add body and prevent separation, various gums were added to the non-aqueous glycerin based formulas. In certain embodiments, carboxymethylcellulose provided the best overall mouthfeel. Carbopol provided body, but in certain embodiments imparted a sticky feel. The formulas were optimized. All lead formulas were stabile at 4 weeks at 40° C.

10% Novamin/20% Pluraflo/CMC (no KCl)

10% Novamin/3.75% KCL/CMC

5% Novamin/3.75% KCL/9% AC43/CMC

### Example 6

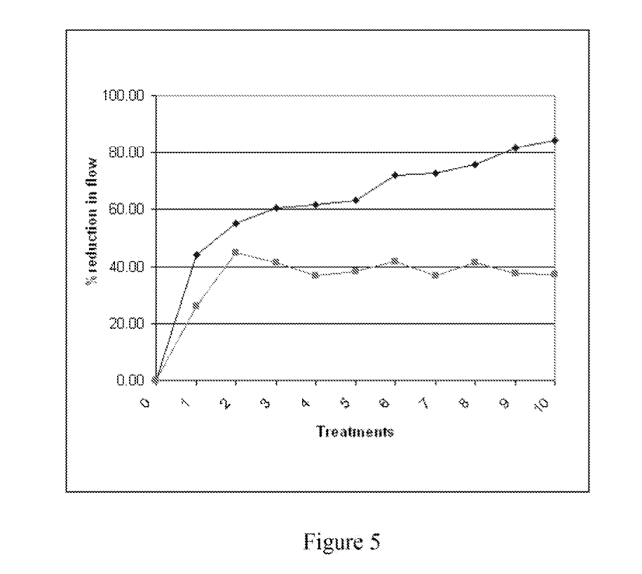

Illustrated in FIG. 5 is a set of conductance data with 10% Novamin toothpaste vs. conventional non-occlusion silica toothpaste control and confocal laser microscopy images showing Novamin dose response and boosting effect of AC43 silica. The top line represents the Novamin sample and the bottom line represents the control sample.

Average Conductance

| | % reduction | | % reduction | |
|---|---|---|---|---|
| Treatments | Novamin 10% | stdev | Control | stdev |
| 0 | 0.00 | 0.00 | 0.00 | 0 |
| 1 | 44.03 | 28.08 | 26.05 | 16.87 |
| 2 | 55.17 | 17.74 | 44.64 | 38.75 |
| 3 | 60.63 | 15.21 | 41.19 | 34.54 |
| 4 | 61.67 | 14.19 | 36.92 | 20.45 |
| 5 | 63.33 | 13.41 | 38.35 | 16.8 |
| 6 | 71.94 | 8.19 | 41.73 | 16.54 |
| 7 | 72.95 | 9.19 | 36.63 | 16.77 |
| 8 | 76.02 | 11.07 | 41.40 | 14.13 |
| 9 | 81.57 | 11.90 | 37.63 | 12.44 |
| 10 | 84.30 | 11.21 | 37.17 | 15.99 |

The invention is not to be limited in scope by the specific embodiments disclosed in the examples, which are intended as illustrations of a few aspects of the invention, and any embodiments, which are functionally equivalent, are within the scope of this invention. Indeed, various modifications of the invention in addition to those shown and described herein will become apparent to those skilled in the art and are intended to fall within the appended claims.

For any references that have been cited, the entire disclosures of which are incorporated herein by reference.

What is claimed:

1. An oral care composition comprising bioactive glass and one or more bioadhesive active components, wherein the oral care composition provides a fluid flow rate of no greater than about 45% of the fluid flow rate of etched dentin.

2. The composition of claim 1, wherein the bioadhesive agent comprises a bioadhesive polymer selected from the group consisting of PEG/PPG copolymers, polyvinylmethylether/maleic anhydride copolymers, polyvinylpyrrolidone (PVP), cross-linked PVP, shellac, polyethylene oxide, methacrylates, acrylates copolymers, methacrylic copolymers, vinylpyrrolidone/vinyl acetate copolymers, polyvinyl caprolactum, polylactides, silicone resins, silicone adhesives, chitosan, milk proteins (casein), amelogenin, ester gum, and combinations thereof.

3. The composition of claim 1 comprising additionally one or more occlusion agents.

4. The composition of claim 3, wherein the occlusion agent comprises arginine/calcium carbonate, arginine bicarbonate/calcium carbonate, and small particle silica or combinations.

5. The composition of claim 2, wherein the bioadhesive agent comprises amino acids including arginine.

4. The composition of claim 1, wherein the one or more bioadhesive polymers comprises from 0.1 wt. % to 70 wt. % by weight of the composition.

5. The composition of claim 1, wherein the one or more bioadhesive polymers comprises from 5 wt. % to 20 wt. % by weight of the composition.

6. The composition of claim 1, wherein the one or more occlusion agents comprises form 0.1% to 50% by weight of the composition.

7. The composition of claim 1, wherein the one or more bioadhesive polymers comprises PEG/PPG copolymers.

8. The composition of claim 1, wherein the one or more bioadhesive polymers comprises polyvinylmethylether/maleic acid.

9. The composition of claim 1, wherein the one or more bioadhesive polymers comprises cross-linked PVP.

10. The composition of claim 1, wherein the one or more bioadhesive polymers comprises shellac.

11. The composition of claim 1, wherein the one or more bioadhesive polymers comprises ester gum.

12. The composition of claim 1, wherein the occlusion agent is a bioactive glass.

13. The composition of claim 1, wherein the occlusion agent is arginine bicarbonate/calcium carbonate.

14. The composition of claim 1, wherein the occlusion agent is a small particle size silica.

15. The composition of claim 1, wherein the composition further comprises an anti-caries agent.

16. The composition of claim 1, wherein the composition further comprises a fluoride source.

17. The composition of claim 1, wherein the composition further comprises an agent to treat xerostomia.

18. The composition of claim 1, wherein the composition further comprises a desensitizing agent.

19. The composition of claim 1, wherein the composition further comprises a whitener or teeth bleach.

20. The composition of claim 1, wherein the composition further comprises an antibacterial agent.

21. An oral care composition comprising an occlusion agent, the occlusion agent comprising:
a. bioactive glass,
b. arginine bicarbonate/calcium carbonate,
c. arginine/calcium carbonate,
d. a small particle silica, and
e. one or more bioadhesive polymer components comprising PEG/PPG copolymers, polyvinylmethylether/maleic acid, cross-linked PVP, shellac, ester gum, and combinations thereof,
wherein the oral care composition provides a fluid flow rate of no greater than about 45% of the fluid flow rate of etched dentin.

22. The composition of claim 21, which is a tooth varnish;

23. A method to treat a tooth comprising applying the composition of claim 21 to a tooth for an effective amount of time.

24. The method of claim 23, wherein the composition is applied to two or more teeth.

25. The method of claim 23, wherein the composition remains on the tooth for at least 1 hour.

26. The method of claim 23, wherein the composition remains on the tooth for at least 2 hours.

27. The method of claim 23, wherein the composition remains on the tooth for at least 5 hours.

28. The method of claim 23, wherein the composition remains on the tooth for at least 24 hours.
